(19) United States
(12) Patent Application Publication
(10) Pub. No.: US 2020/0093521 A1
(43) Pub. Date: Mar. 26, 2020

(54) ROD REDUCER

(71) Applicant: Astura Medical Inc., Carlsbad, CA (US)

(72) Inventors: Keith Klausman, Carlsbad, CA (US); Thomas Purcell, Carlsbad, CA (US)

(73) Assignee: Astura Medical Inc., Carlsbad, CA (US)

(21) Appl. No.: 16/581,736

(22) Filed: Sep. 24, 2019

Related U.S. Application Data
(60) Provisional application No. 62/735,828, filed on Sep. 24, 2018.

(51) Int. Cl. A61B 17/70

(52) U.S. Cl. CPC A61B 17/7086; A61B 17/7032

(57) ABSTRACT

A rod reducer designed to deliver a set screw by way of a decoupled ram and driver shaft. The driver shaft secures and delivers the set screw while the ram applies the load to the rod for reduction. By separating the ram and driver, the rod is reduced down below the start of the female threads, allowing for the set screw to be started by the driver, avoiding binding and additional steps.

### CROSS-REFERENCE TO RELATED APPLICATIONS

This application claims the benefit of U.S. Provisional Application No. 62/735,828 filed Sep. 24, 2018, which is incorporated herein by reference.

### FIELD

The present invention relates generally to the field of surgery, and more specifically, to a rod reducer for use in spinal fusion surgery.

### BACKGROUND

Many spinal fixation systems use pedicle screws attached to two or more vertebrae coupled to a fixation rod. The pedicle screw includes a body member that includes a U-shaped channel or slot to accept the fixation rod. A set screw is used to threadably engage the body member of the screw assembly to secure the stabilizing rod within the body member. Positioning the fixation rod in the screw head typically requires the drawing of the rod to the screw using a rod reducer.

Rod reducers usually contact the set screw first, and which then pushes the set screw into the fixation rod during rod reduction. Pushing the set screw in this manner may lead to set screw binding, cross threading, additional counter clockwise rotation steps, and or unwanted downward force to engage into female thread.

Some prior art devices us counter clockwise rotation to align start of male threads with female threads. The counter clockwise rotation for alignment fails due to the load required to be placed on the set screw which in turn is applied to the female threads, producing binding and leading to thread deformation and cross threading.

Thus, there is a need for an improved rod reducer that solves the problems listed above.

### SUMMARY

The present invention is directed to a rod reducer that applies the reduction load to the rod instead of the set screw. This is accomplished by using a ram that is decoupled from the driver shaft. The driver shaft secures and delivers the set screw while the ram applies the load to the rod for reduction. By separating the ram and driver, the rod is reduced down below the start of the female threads in the screw extender, allowing for the set screw to be started by the driver, avoiding binding and additional steps.

### BRIEF DESCRIPTION OF THE DRAWINGS

FIG. 1 is a view showing a one embodiment of a rod reducer.

FIG. 2 is a perspective view showing the distal end of the rod reducer of FIG. 1.

FIG. 3 is a perspective view showing the rod reducer of FIG. 1 in use with a pedicle screw, screw extender and fixation rod.

FIG. 4 is an enlarged perspective view showing the ram of the rod reducer engaging the fixation rod.

FIG. 5 is an enlarged perspective view showing the driver shaft engaging the set screw.

FIGS. 6A-6C are views showing features of the handle for individual actuation of the ram and drive shafts.

### DETAILED DESCRIPTION

The present invention is a rod reducer having decoupled shafts, with a first shaft being a ram configured to reduce a fixation rod into a receiving channel of a pedicle screw head, and the second shaft being a driver shaft configured to advance a set screw to secure the fixation rod. In the embodiments shown, the ram applies the load to the rod for reduction and the driver shaft secures and delivers the set screw. By separating the ram and driver shaft, the rod is reduced down below the start of the female threads, allowing for the set screw to be started by the driver shaft after reduction of the rod, avoiding binding and additional steps.

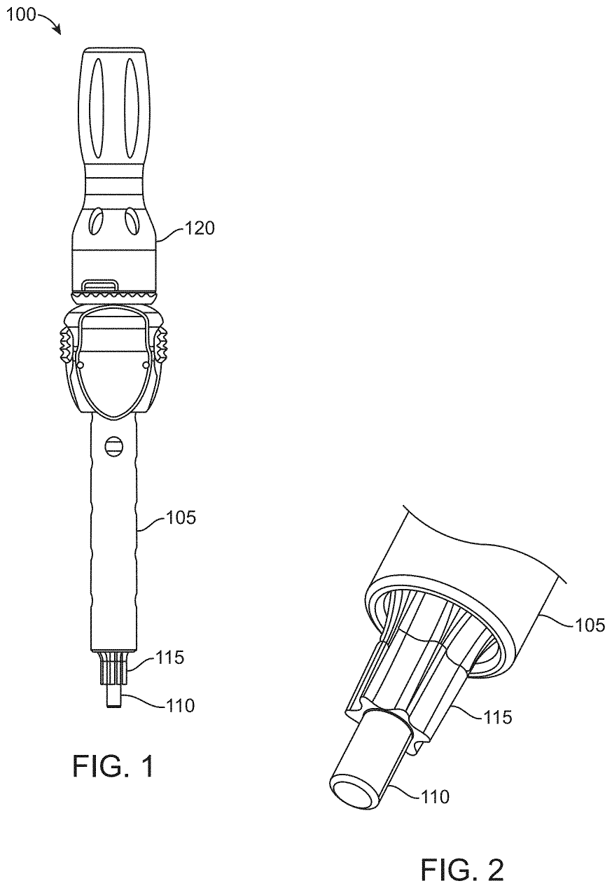

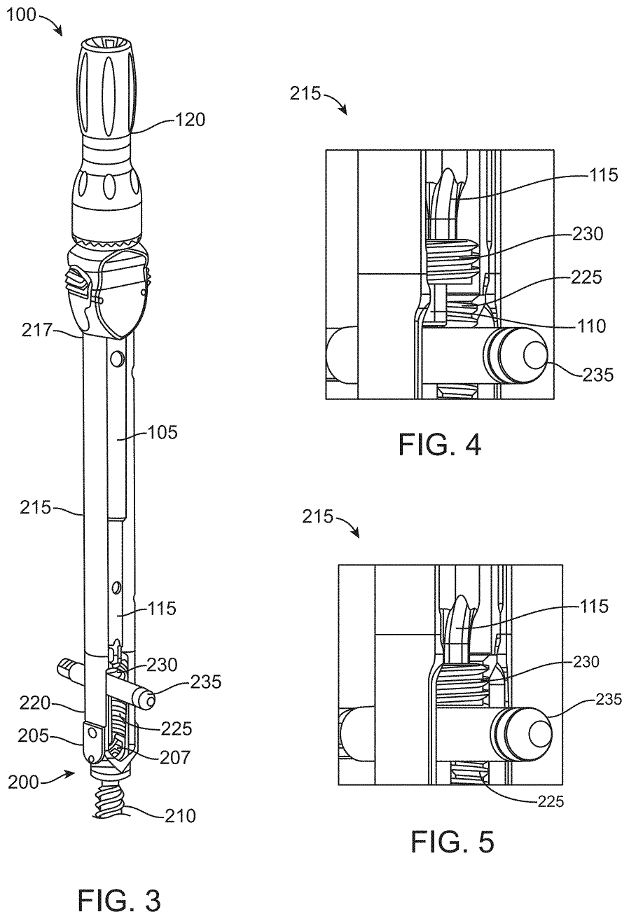

FIG. 1 shows one embodiment of a rod reducer 100 having a body 105, a ram 110, driver shaft 115 and a handle 120. The ram 110 and driver shaft 115 are positioned within the body 105 and are configured to operated independently relative to one another. In one embodiment, the ram 110 is configured to apply a load to the fixation rod for reduction, and the driver shaft 115 is configured to advance a set screw to secure the fixation rod to the pedicle screw head (see FIGS. 3-5). In other embodiments, the ram 110 and/or driver shaft 115 may be configured to do additional actions.

FIG. 2 shows one embodiment of the rod reducer in which the ram 110 is positioned within a central lumen of the driver shaft 115. In a first position, a ram distal end 110a extends distally beyond a driver distal end 115a. This position allows the ram distal end 110a to be the first contact with the fixation rod and apply the load to reduce the fixation rod to a second position below the start of female threads in the screw extender (see FIGS. 4 and 5). In this second position, the set screw contacts the female threads and the driver shaft 115 rotates and advances the set screw in the female threads. Allowing for the set screw to be started by the driver shaft 115 after reduction of the rod to a position below the female threads avoids the problems of the prior art systems using only the set screw for both the reduction load for the fixation rod and advancing the set screw. securing the fixation rod. load and also trying to secure it within the pedicle screw head at the same time.

FIGS. 3-5 show the rod reducer 100 in use with a pedicle screw 200 having a proximal head portion 205 and distal screw portion 210 and a screw extender 215. The head portion 205 includes a U-shaped channel 217 with internal treads configured to receive the fixation rod 235 and set screw 230. The distal screw portion 210 is configured for insertion into a bone (not show). The screw extender 215 is used to assist in guiding of the fixation rod 235 and set screw 230. The screw extender 215 includes a distal end 220 configured to couple to the screw head 205, and a proximal end 217 configured to couple with the body 105. The distal end 220 also includes a distal threaded portion 225 having internal female threads 225 configured to receive the set screw 230. The screw extension 215 includes a slot sized to receive the fixation rod 235. The set screw 230 is positioned within the screw extender 215, above the fixation rod 235. The set screw 230 is configured to engage female threads 225 of both the screw extender 215 and head portion 217.

The body 105 is inserted into the screw extender 215, the body 105 having various attachment features known in the art to engage and couple with the screw extender 215. For example, the body 105 may be configured to couple with mating threads on the proximal end of the screw extender 215.

The rod reducer 100 includes various features, such as the ram 110, to reduce the fixation rod 235 into a receiving portion 207 of the screw head 205 and a driver shaft 115 to insert the setscrew 230 into the receiving portion 2017 to retain the fixation rod 2235 therein.

As illustrated in FIG. 3, the body 105 of the rod reducer 100 may be inserted into the screw extender 215 and coupled at its proximal end 217. A distal end of the screw extender 220 couples with the receiving portion 205 of the screw assembly 200. The handle 120 attaches to the proximal end of the rod reducer 100 and includes features to actuate the ram 110 and driver shaft 215 of the rod reducer 100. The fixation rod 235 may be inserted into the distal end of the screw extender 220 while the rod reducer 100 is in a retracted position (see FIG. 4). The fixation rod 235 is advanced distally by the ram 110 as the rod reducer 100 moves towards the extended or second position where the fixation rod 235 is below the start if the internal female threads 225 of the screw extender 215 (see FIG. 5). Once the fixation rod 235 is reduced below the internal female threads 225 of the screw extender 215, the handle 120 may include a second actuating feature for actuating the driver shaft 215 to advance the set screw 230 into the female threads, and may continue advancing until the set screw 235 is in the receiving portion 207 of the screw head 205 to rigidly couple the fixation rod 235.

The rod reducer 100 is designed to reduce the fixation rod 235 within the screw assembly 215, first using the ram 110 to move the fixation rod 235 below the female threads 225, then using the driver shaft 115 to advance the set screw 230 in threaded engagement with the female threads 225 on the screw extender 215. The handle 120 is configured to operate the ram 110 and driver shaft 115.

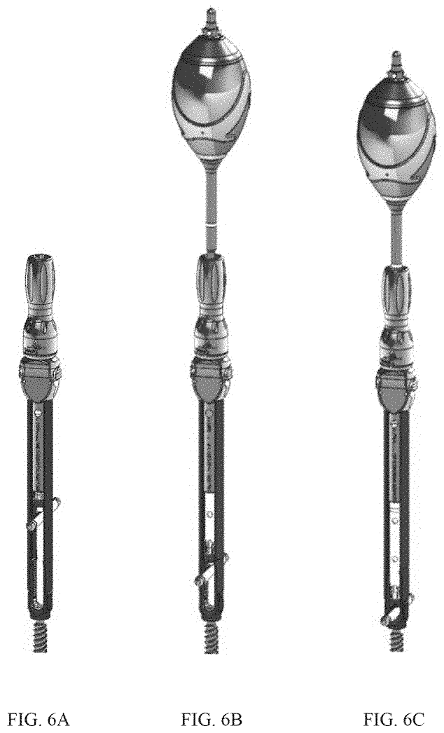

FIGS. 6A-6C illustrate features of the handle 120 for individual actuation of the ram 110 and drive shafts 115. The knob drives the ram downward for the first 30mm approximately. Once the rod is seated below the start of the threads, the Reducer Driver engages the drive shaft and threads down the set screw

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. A rod reducer comprising:
a body having a lumen;
a ram configured to apply a load to the fixation rod for reduction;
a driver shaft configured to advance a set screw to secure the fixation rod to the pedicle screw head
the driver shaft having an axial lumen and the ram being configured to fit within the driver shaft lumen, the driver shaft being configured to fit within the body lumen;
a handle coupled to the ram and driver shaft near a proximal end of the body;
wherein the ram and driver shaft being configured to operated independently relative to one another.
